(19) United States
(12) Patent Application Publication
(10) Pub. No.: US 2022/0249268 A1
(43) Pub. Date: Aug. 11, 2022

(54) KNEE ORTHOSIS

(71) Applicant: OTTOBOCK SE & CO. KGAA, Duderstadt (DE)

(72) Inventors: Olaf KROLL-ORYWAHL, Northeim (DE); Gordon SIEWERT, Göttingen (DE)

(73) Assignee: OTTOBOCK SE & CO. KGAA, Duderstadt (DE)

(21) Appl. No.: 17/628,884

(22) PCT Filed: Jul. 21, 2020
(86) PCT No.: PCT/EP2020/070531

§ 371 Date: Jan. 20, 2022

(30) Foreign Application Priority Data
Jul. 25, 2019 (FR) 10 2019 120 181.0

(51) Int. Cl. A61F 5/01

(52) U.S. Cl. CPC A61F 5/0123; A61F 2005/0197; A61F 2005/0172; A61F 2005/0167

(57) ABSTRACT

The invention relates to a knee orthosis with an upper leg frame for arranging on an upper leg and a lower leg frame for arranging on a lower leg, wherein the lower leg frame is arranged on the upper leg frame such that it can be swivelled about a first swivel axis by means of at least one joint, and a calf pad for mounting on a calf, wherein the knee orthosis comprises at least one elastic bearing element that is attached in such a way that the bearing element is tensioned when the knee orthosis is mounted and, in this state, presses the calf pad either directly or indirectly against a calf of the wearer of the knee orthosis.

The invention relates to a knee orthosis with an upper leg frame for arranging on an upper leg and a lower leg frame for arranging on a lower leg, wherein the lower leg frame is arranged on the upper leg frame such that it can be swivelled about a first swivel axis by means of at least one joint, and a calf pad for placing on a calf.

Such knee orthoses have been known from the prior art for many years and used particularly after operations on the posterior cruciate ligament so as to prevent the so-called “posterior drawer”. Particularly following a rupture to the posterior cruciate ligament and the subsequent operation, the lower leg may slip backwards relative to the upper leg, as the injury has caused an instability in the knee. This behavior has been known for many years and attempts made to resolve it using knee orthoses.

For example, EP 1 575 464 B1 discloses a knee orthosis according to the preamble whose calf pad is designed as a half-shell that is arranged around the calf. The half-shell has an end close to the knee and an end far from the knee, each of which is arranged on a splint, both splints being connected to the swivel joint between the upper leg frame and the lower leg frame. A spring is arranged between the two splints through which a force is exerted that is intended to reduce the angle between the two splints. A torque is thus applied to the calf pad, designed as a half-shell, which causes the part of the half-shell close to the knee to be pushed forward. However, the disadvantage is that this torque can create a situation that is perceived as unpleasant. The half-shall can also lose contact with the calf, especially when the knee is flexed, i.e. strongly bent, so that the desired effect does not occur or no longer occurs completely.

Knee orthoses are also known from the prior art that feature a Bowden cable, by way of which a bending or flexion of the knee is converted into an applied pressure that is applied to the calf by the calf shell. Such constructions are structurally complex and therefore expensive; they are also prone to error and require a lot of maintenance. In addition, all knee orthoses of the type described here that are known from the prior art have the disadvantage that the force applied by the calf pad cannot be adjusted, such that reproducibility cannot be achieved to the desired extent.

The invention therefore aims to propose a structurally simple knee orthosis by means of which the applied force can be applied in a manner that is comfortable for the wearer of the knee orthosis and reproducibility of the applied force is achieved.

The invention solves the problem by way of a knee orthosis according to the preamble of claim 1, which is characterized in that the knee orthosis comprises at least one elastic bearing element that is attached in such a way that the bearing element is tensioned when the knee orthosis is mounted and, in this state, presses the calf pad either directly or indirectly against a calf of the wearer of the knee orthosis.

The knee orthosis according to the invention thus features an upper leg frame for arranging on an upper leg. Preferably, at least one strap or belt is placed around the upper leg to fix the upper leg frame to the upper leg. The frame itself preferably features a medial splint and a lateral splint that are connected to each other by the specified strap, for example. The knee orthosis according to the invention also features a lower leg frame that can be arranged on a lower leg. Preferably, this can also be attached to the lower leg via at least one strap or belt. The lower leg frame preferably has a medial splint and a lateral splint that are connected to each other. The lower leg frame and upper leg frame are connected to each other by means of at least one joint. This is preferably a swivel joint that permits a swivelling of the upper leg frame relative to the lower leg frame about a first swivel axis. The lower leg frame and upper leg frame are preferably connected to each other by two joints whose swivel axes coincide. Here, one joint is preferably arranged medially and connects the medial splint of the upper leg frame to the medial splint of the lower leg frame. The second joint is preferably arranged laterally and connects the lateral splint of the upper leg frame to the lateral splint of the lower leg frame.

The at least one elastic bearing element acts as an elastic energy store, which is also referred to as a mechanical energy store. When mounting the knee orthosis, the at least one elastic bearing element is tensioned. During this process, mechanical energy, particularly deformation energy, is stored in the bearing element. The at least one elastic bearing element then exerts a force that directly or indirectly presses the calf pad against the calf of the wearer of the orthosis. Specifically, this means that the bearing element directly or indirectly exerts a force on the calf pad that acts in the direction of the calf of the wearer. This may also be a tensile force that is directly or indirectly exerted on the calf pad.

The knee orthosis preferably has a pad retainer, which is arranged on the lower leg frame. The pad retainer may be, for example, a plastic element, for example made of a rigid plastic, or a metal element. The pad retainer may also be in the form of a preferably inelastic yet flexible element, such as a textile strap. The pad retainer is preferably arranged on the medial splint and lateral splint of the lower leg frame. It is thus preferably connected to the lower leg frame at at least two points. This increases stability.

The at least one elastic bearing element is preferably arranged on the pad retainer. It is preferably located on a side of the pad retainer facing the calf and fixed to it. The at least one elastic bearing element can be detachably connected to the pad retainer or such that it cannot be detached. The calf pad is arranged on the at least one elastic bearing element, the calf pad being positioned towards the calf. It is preferably padding or a cushion, for example, to ensure the comfort of the wearer of the knee orthosis. If the knee orthosis is mounted, the upper leg frame is arranged on the upper leg and the lower leg frame on the lower leg of the wearer, so that the first swivel axis almost coincides, but preferably does so as precisely as possible, with the swivel axis of the knee of the wearer of the knee orthosis. The calf pad is arranged on the wearer's calf and comes into contact with it. The geometric design and/or length of the pad retainer ensures that the at least one elastic bearing element is tensioned when the knee orthosis is mounted. As it is an elastic bearing element, this generates a restoring force that counteracts the tension. As a result, a compression force is exerted on the calf of the wearer of the knee orthosis by the calf pad.

Preferably, the at least one elastic bearing element in the tensioned state exerts a frontal-acting force on the calf pad that is transferred to the wearer's calf by the calf pad. Unlike in some embodiments of a knee orthosis known from the prior art, there is no torque applied to the calf pad in this embodiment that is transferred to the lower leg of the wearer; rather, the at least one elastic bearing element generates a force that acts in a single direction, preferably frontally, and thus pushes the lower leg forwards.

The pad retainer can preferably be attached to the lower leg frame in multiple different positions and/or orientations relative to the lower leg frame. This may occur in various ways. For example, if the pad retainer is a plastic or metal element, it may feature a plurality of positive-locking elements, with correspondingly designed positive-locking elements being provided on the lower leg frame in this case. This can be achieved, for example, in the form of press studs so that the pad retainer can be arranged in different positions on the lower leg frame. In this way, it is particularly easy to adjust the effective length of the pad retainer, i.e. the distance between the two fastening elements on which the pad retainer is attached to the lower leg frame. This distance is measured along the pad retainer. The positive-locking elements may also be velcro elements, which enables the position and/orientation of the pad retainer relative to the lower leg frame to be infinitely changed.

If the pad retainer is a strap or belt, it may feature a plurality of holes, for example, which enable it to be fastened to clasps provided for such a purpose, which are arranged on the lower leg frame, for example. In this embodiment, the effective length of the pad retainer can be adjusted by selecting the respective hole by means of which it is to be fastened to the clasp.

In these embodiments, the effective length of the pad retainer determines how strongly the at least one elastic bearing element is tensioned, preferably compressed, when mounting the knee orthosis. The shorter the effective length, the more strongly the at least one elastic bearing element is tensioned when the knee orthosis is mounted. As a result, the force transferred by the at least one elastic bearing element to the calf pad and by said calf pad to the wearer's lower leg is changed. By changing the position and/or orientation of the pad retainer relative to the lower leg frame, the force exerted on the calf and the lower leg in the mounted state becomes adjustable.

In a preferred embodiment, the position and/or orientation of the pad retainer relative to the lower leg frame is infinitely adjustable and the pad retainer can be fixed in every one of these infinitely adjustable positions and/or orientations. This is partitularly easy to achieve if velcro elements are arranged on the pad retainer and velcro counter-elements are arranged on the lower leg frame, so that the pad retainer can be easily detached from the lower leg frame and reattached to it in the desired position and/or orientation.

Preferably, the calf pad is detachably arranged on the pad retainer. This can also preferably be achieved via positive-locking elements, such as press studs, velcro elements or other elements. It is therefore possible to remove the calf pad from the pad retainer and, for example, to clean it or replace it with another calf pad. This may be practical, for example, if the calf pad is not suited to the individual requirements of the wearer of the knee orthosis, as it is too large or small, for example, or a different degree of padding is desired. In this way, it is also possible to reuse the remaining components of the knee orthosis, so that not every patient requires their own knee orthosis of the type described here. This is an advantage because the knee orthosis is often not used for a long period of time, but for a limited time, for example following a surgical operation.

The at least one elastic bearing element preferably has a compression element, for example a compression spring and/or a component made of an elastic material. For example, it may be made of rubber or an elastomer, wherein the actual elasticity of the at least one elastic bearing element not only depends on the material of the compression element, but also its geometry. Preferably, more than one elastic bearing element is arranged on the pad retainer, which ensures that multiple elastic bearing elements are compressed when mounting the knee orthosis and a counterforce is thus exerted on the calf pad resting on the wearer's lower leg. This enables a more homogeneous distribution of force, which can increase the level of comfort felt when wearing the orthosis and the therapeutic effect. In addition, the already good auto-adaptation and auto-adaptive adaptation of the shape and contour of at least one calf pad to the wearer's calf and lower leg is further improved.

Preferably, the at least one elastic bearing element has at least one tension element, preferably a tension spring and/or at least one elastic strap. The calf pad is preferably arranged on another component of the knee orthosis, for example the lower leg frame, by at least one, preferably two such elastic straps. The bearing element is tensioned during mounting so that energy is stored. Here, a bearing element designed as an elastic strap is expanded. An expansion limiter is preferably provided, which is designed, for example, in the form of an inelastic strap or strap section, which is arranged parallel to the elastic strap. “Parallel” does not mean parallel in geometrical terms, but parallel with regard to the effectiveness of the two straps or strap sections, i.e. in particular not arranged one behind the other or in series.

In a preferred embodiment, the at least one calf pad is arranged on the pad retainer such that it can be swivelled about at least a second swivel axis, wherein the second swivel axis is preferably at least almost, but especially preferably completely, vertical to the first swivel axis. This renders it possible, for example, to optimally adjust the orientation of the calf pad to the individual conditions of the wearer's calf and lower leg by means of another swivel joint or ball joint. Ideally, this adaptability is achieved when several swivel joints coupled together, a cardan joint or a ball joint are used. As a result the calf pad is mounted on the pad retainer via the at least one elastic bearing element such that it can be swivelled in all directions.

The knee orthosis preferably features a measurement scale on which a strength of the compression of the at least one elastic bearing element and/or a force generated by this tension can be read. This ensures that the force generated by the tensioned, at least one elastic bearing element can be defined, so that it is possible to adjust it to a desired value and to subsequently check whether the generated force still has the desired strength. This is rendered particularly easy by the use of the measurement scale, so that it can be done by the patient without the help of an orthopedic technician. This further reduces the effort required when using the knee orthosis, which increases acceptance.

In the following, some examples of embodiments of the present invention will be explained in more detail by way of the attached figures: They show:

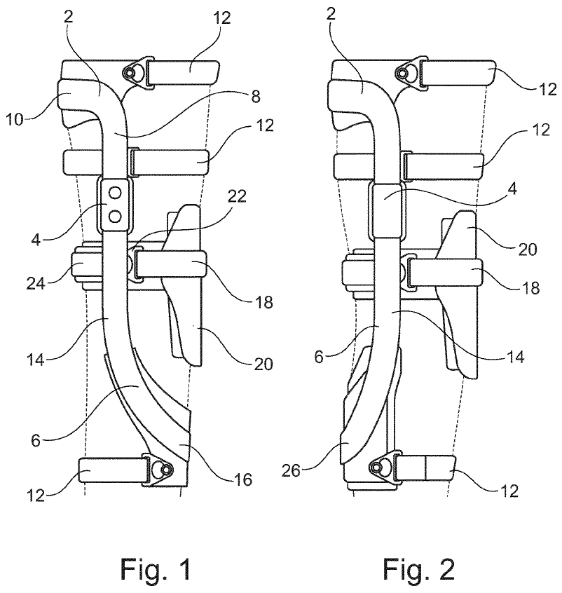

FIGS. 1 to 2—schematic side representations of two different embodiments of knee orthoses according to examples of embodiments of the present invention,

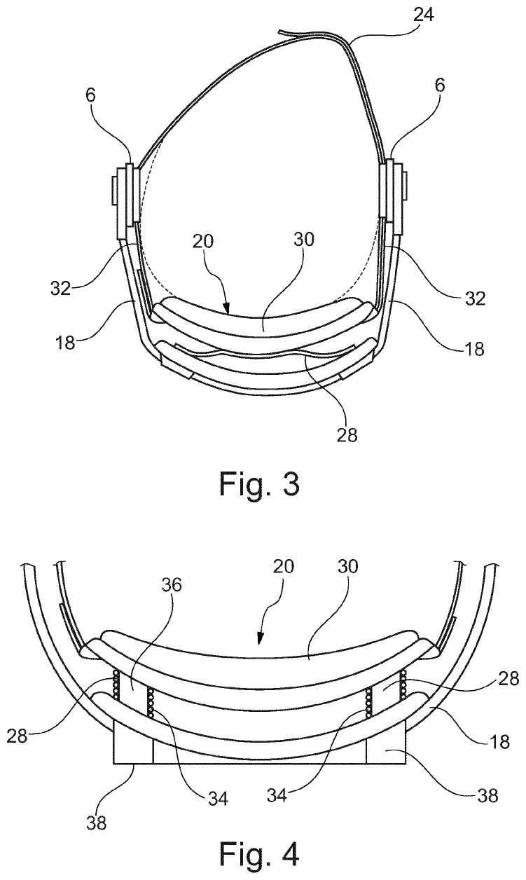

FIG. 3—the schematic representation of a top view of a knee orthosis according to an example of an embodiment of the present invention,

FIGS. 4 to 6—a top view of enlarged sections of knee orthoses according to further examples of embodiments of the present invention,

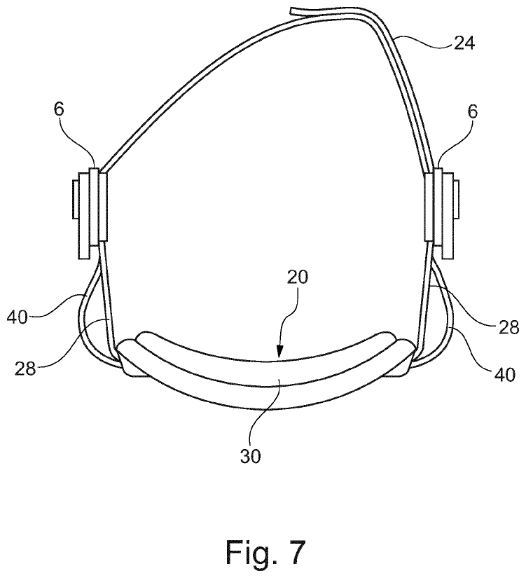

FIG. 7—a section from a knee orthosis according to a further example of an embodiment.

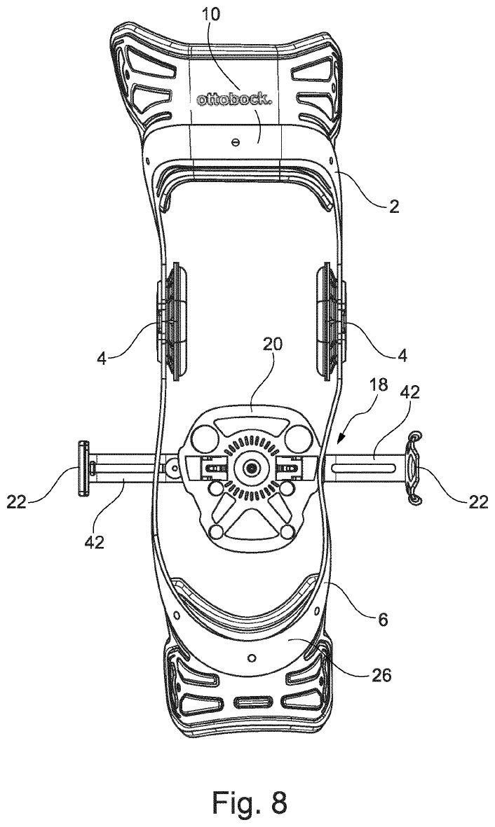

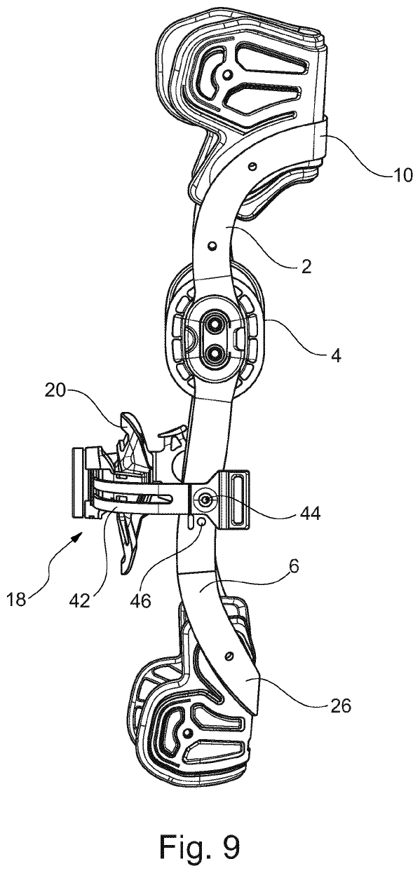

FIGS. 8 and 9—schematic representations of a further knee orthosis according to an example of an embodiment of the present invention,

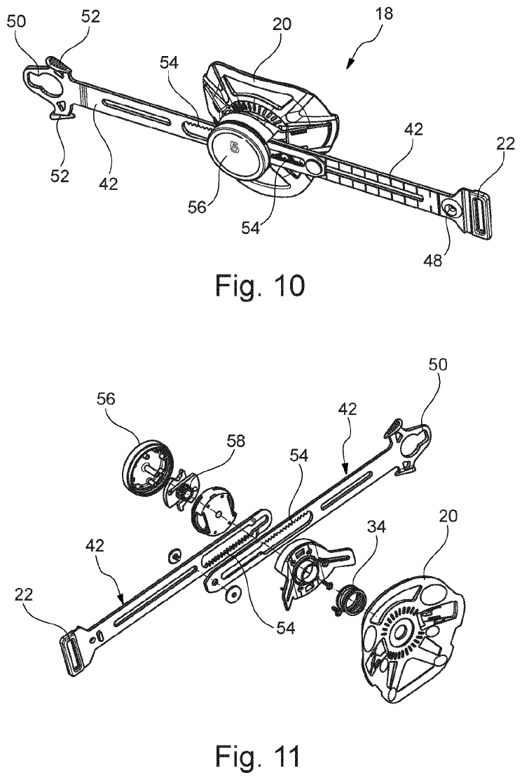

FIG. 10—a component of the orthosis from FIGS. 8 and 9, and

FIG. 11—an exploded view of the component from FIG. 10.

FIG. 1 shows a knee orthosis according to a first example of an embodiment of the present invention when in the mounted state. It has an upper leg frame 2 that is connected to a lower leg frame 6 via a joint 4. The upper leg frame 2 features a medial splint 8 and a lateral splint, not depicted in the side view in FIG. 1, that is connected to the medial splint 8 via a frontal connecting element 10. In the example of an embodiment shown, the medial splint 8, the lateral splint—not shown—and the frontal connecting element 10 are designed as a single-piece component. The upper leg frame 2 is arranged on the wearer's upper leg via two straps 12.

The lower leg frame 6 also features a medial splint 14 and a lateral splint not depicted in FIG. 1. The medial splint 14 and the lateral splint are connected to each other via a dorsal connecting element 16. In the embodiment according to FIG. 1, the medial splint 14, the lateral splint—not depicted—and the dorsal connecting element 16 are designed as a single piece. The thus-formed lower leg frame 6 is fixed to the lower leg via a strap 12.

A pad retainer 18 is also arranged on the lower leg frame, a calf pad 20 in the form of a half-shell being located on said pad retainer in the example of an embodiment shown. The pad retainer 18 is arranged on the lower leg frame 6 via two connecting elements 22, only one of which is depicted in FIG. 1. An additional strap 24 is also provided at this point, which ensures increased stability.

FIG. 2 depicts another configuration of a knee orthosis according to a further example of an embodiment of the present invention. It also features the upper leg frame 2, which is arranged on the wearer's upper leg via two straps 12 and connected to a lower leg frame 6 via a joint 4. Unlike the example of an embodiment shown in FIG. 1, the medial splint 14 and the lateral splint—not depicted—of the lower leg frame 6 are connected to each other via a frontal connecting element 26. The lower leg frame 6 is fixed to the lower leg via a strap 12. As already demonstrated in the example of an embodiment shown in FIG. 1, the pad retainer 18 with the calf pad 20 located on it is also arranged on the lower leg frame 6.

FIG. 3 depicts a schematic top view of a knee orthosis according to an example of an embodiment of the present invention. For the sake of clarity, the upper leg frame has been omitted. The pad retainer 18 can be seen, which is arranged on elements of the lower leg frame 6 that are only schematically depicted. It extends dorsally around the wearer's leg. The strap 24 is located at the front and is preferably adjustable in length and can be opened to mount the knee orthosis.

An elastic bearing element 28 is schematically depicted on the pad retainer 18, on which the calf pad 20 is located. This features a cushion 30 that can preferably be taken out for cleaning or for replacing with another cushion. When mounting the knee orthosis, the strap 24 is opened and the leg of the wearer of the orthosis introduced into the orthosis. The elastic bearing element 28 is compressed during this process. Specifically, this means that the calf pad 20 is displaced towards the pad retainer 18, so that a distance between the calf pad 20 and the pad retainer 18 is reduced. Since the elastic bearing element 28 is located in this gap, it is compressed when the knee orthosis is mounted.

In the example of an embodiment shown, the at least one elastic bearing element 28 is designed in the form of a leaf spring and may be made of a metal or a plastic, for example. Due to the elasticity of the elastic bearing element 28, the compression of the bearing element 28 results in a counter-force that presses the calf pad 20 against the wearer's calf. In the example of an embodiment shown, the calf pad 20 is arranged on the lower leg frame 6 via fastening straps 32 in order to ensure sufficient stability and mechanical strength.

FIG. 4 shows an enlarged section from a top view of a knee orthosis according to a further example of an embodiment. The pad retainer 18 and the calf pad 20 with the cushion 30 are visible, the calf pad being mounted on the pad retainer 18 via elastic bearing elements 28 in the example of an embodiment shown. The respective elastic bearing element 28 comprises an elastically compressible spiral spring 34 and a guide pin 36, which is mounted in a pin accommodation 38 such that it can be displaced. As previously explained, when the knee orthosis is mounted, the calf pad 20 is displaced towards the pad retainer 18. The guide pins 36 are inserted into the pin accommodation 38 and the spiral spring 34 simultaneously compressed.

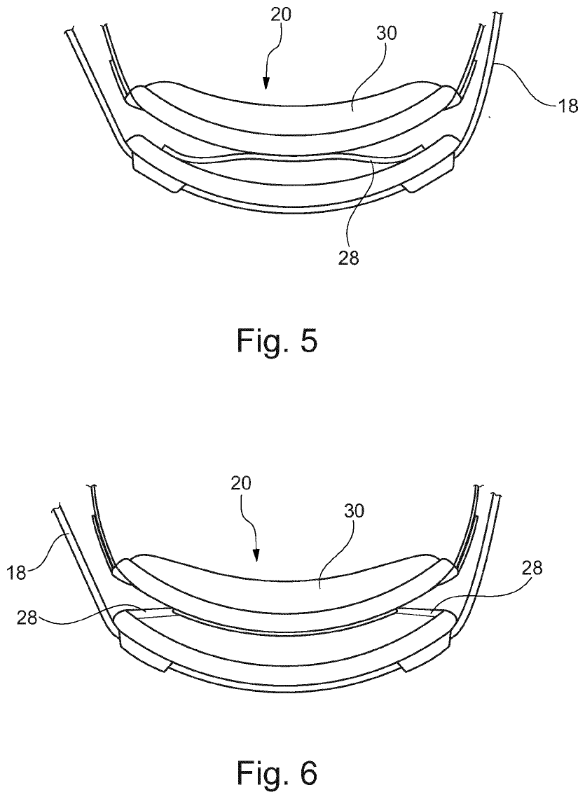

FIGS. 5 and 6 depict two further embodiments, each of which feature different elastic bearing elements 28 via which the respective calf pad 20 with the cushion 30 is arranged on the respective calf retainer 18. FIG. 5 depicts the elastic bearing element 28 that is already shown in FIG. 3 and comprises a leaf spring. In FIG. 6, two elastic bearing elements 28 are provided that are designed as elastic projections or tabs and on which the calf pad is arranged.

FIG. 7 shows another embodiment. Again, the calf pad 20 features the cushion 30 and is arranged on the lower leg frame 6. Unlike the examples of embodiments depicted thus far, in the embodiment shown in FIG. 7 the elastic bearing elements 28 are designed as tension elements. In the example of an embodiment shown, the bearing elements 28 are elastic straps, by means of which the calf pad 20 is arranged on the lower leg frame 6. When mounting the knee orthosis, the elastic bearing elements 28 are tensioned, i.e. expanded in the example of an embodiment shown, so that mechanical energy is stored. As a result, a force acting upwards in FIG. 7 is applied to the calf pad 20, so that the calf pad 20 remains in contact with a calf of the wearer, not depicted. Two expansion limiters 40 are depicted parallel to the bearing element 28, said limiters being made of an inelastic strap section. If the bearing elements 28 are expanded too strongly, they reach the length of the inelastic strap sections of the expansion limiters 40 and further expansion is not possible.

The knee orthosis shown is also arranged on the wearer's lower leg via the strap 24 in this embodiment.

FIG. 8 depicts a frontal view of a knee orthosis according to a further example of an embodiment of the present invention. It features two frontal connecting elements 10, 26, one of which forms part of the upper leg frame 2 and the other part of the lower leg frame 6. The knee orthosis has two joints 4 via which the lower leg frame 6 is arranged on the upper leg frame 2 such that it can be swivelled. The calf pad 20 is attached to the lower leg frame 6 via a pad retainer 18. A cushion, not depicted in FIGS. 8 and 9, can be arranged on the calf pad 20 to create a padding. In the example of an embodiment shown, the pad retainer 18 features two arms 42, at each end of which the connecting element 22 is arranged, with which the respective arm 42 can be attached to the lower leg frame 6. In the representation shown in FIG. 8, the arms 42 are not attached.

FIG. 9 shows the knee orthosis from FIG. 8 in a side view from the medial or lateral perspective. The upper leg frame 2 is connected to the lower leg frame 6 via the joint 4. The two frontal connecting elements 10, 26 are designed to be arranged on the upper leg and lower leg. On the lower leg frame 6 one of the arms 42 can be seen, the arms being attached to the lower leg frame 6 via a fastening pin 44, wherein the fastening pin 44 is guided through one of several holes 46 provided in the example of an embodiment shown. As depicted in FIG. 9, the arm 42 is bent in the process. It is designed to be elastic, or at least flexible. The calf pad 20 is arranged on the pad retainer 18 to which the two arms 42 belong, wherein said calf pad can be replaced with a cushion, not depicted, as previously explained. It is hinged via an elastic bearing element 28, which is not shown in FIG. 9. This elastic element 28 can be a compression spring, for example, which presses the calf pad 20 in FIG. 9 to the right. Consequently, if the knee orthosis is mounted on a knee in accordance with FIG. 9, the leg and in particular the calf presses the elastic bearing element 28 together so that a force is applied to the calf pad 18.

FIG. 10 shows the pad retainer 18 used in the orthoses according to figures9 and 8. It features the two arms 42, which protrude laterally from the calf pad 20. On the arm 42 depicted to the right in FIG. 10 is a recess 48 through which a fastening pin 44 is guided in order to attach the arm 42 to the lower leg frame 6. A connecting element 22 is located next to it on which, for example, a fastening strap 32 can be arranged. Such a fastening strap 32, not depicted, can enable a further increase in stability and allow the knee orthosis to be arranged more securely on the wearer's leg.

A positive-locking element 50 is located on the arm 42 to the left in FIG. 10 that can interact with a correspondingly designed positive-locking element arranged on the lower leg frame 6 in such a way that the arm 42 can be arranged especially easily on the lower leg frame 6. This results in a positive-locking connection of the two positive-locking elements. If the knee orthosis is mounted in accordance with FIGS. 8 and 9, the positive-locking element 50 is preferably released from the correspondingly designed positive-locking element on the lower leg frame 6 before the knee orthosis is mounted. Only once the upper leg frame 2 is resting on the upper leg and the lower leg frame 6 on the lower leg is the positive-locking element 50 connected to the correspondingly designed positive-locking element on the lower leg frame 6. To this end, the end of the arm 42 features grip elements 52 that render it particularly easy for the wearer of the orthosis to grip the end of the arm 42 and connect it to the positive-locking element on the lower leg frame 6.

The two arms 42 are each fitted with a row of teeth 54 that engages in the positive-locking element, not depicted. Preferably, a snap connection is formed between one of the arms 42 and one of the positive-locking elements. In a preferred embodiment, the cap 56 can be rotated to release this snap connection. In this position, at least one arm 42, but preferably each arm 42, can be displaced relative to the calf pad 20, so that the effective length of the pad retainer 18 can be adjusted. It is therefore possible to adjust how strongly the elastic bearing element 28, not depicted, is compressed by the wearer's calf, so that the force acting on the calf is adjustable. The effective length of the pad retainer 18 is preferably the distance between the recess 48 and the positive-locking element 50.

FIG. 11 shows the component from FIG. 10 in an exploded view. The two arms 42 can be recognized, each of which features a row of teeth 54 and which are designed so that they can be displaced relative to each other. A gear wheel 58, among other things, is located between the arms 42 and the cap 56, said gear-wheel engaging with the two rows of teeth 54. In this way, a length, which corresponds to the distance between the connecting element 22 and the positive-locking element 50, is locked once set. The actual calf pad 20 is elastically mounted via the spiral spring 34.

### REFERENCE LIST

2 upper leg frame

4 joint

6 lower leg frame

8 medial splint

10 frontal connecting element

12 strap

14 medial splint

16 dorsal connecting element

18 pad retainer

20 calf pad

22 connecting element

24 strap

26 frontal connecting element

28 elastic bearing element

30 cushion

32 fastening strap

34 spiral spring

36 guide pin

38 pin accommodation

40 expansion limiter

42 arm

44 fastening pin

46 hole

48 recess

50 positive-locking element

52 grip element

54 row of teeth

56 cap

58 gearwheel

1. A knee orthosis with
an upper leg frame for arranging on an upper leg and
a lower leg frame for arranging on a lower leg,
wherein the lower leg frame is arranged on the upper leg frame such that it can be swiveled about a first swivel axis by means of at least one joint, and
a calf pad for mounting on a calf, wherein the knee orthosis comprises at least one elastic bearing element that is attached in such a way that the at least one bearing element is tensioned when the knee orthosis is mounted and, in this state, presses the calf pad either directly or indirectly against a calf of a wearer of the knee orthosis.

2. The knee orthosis of claim 1, wherein the knee orthosis features a pad retainer arranged on the lower leg frame, wherein the calf pad is arranged on the pad retainer via the at least one elastic bearing element.

3. The knee orthosis of claim 1, wherein the at least one elastic bearing element in the tensioned state exerts a frontal-acting force on the calf pad.

4. The knee orthosis of claim 2, wherein the pad retainer can be fixed on the lower leg frame in multiple positions and/or orientations relative to the lower leg frame.

5. The knee orthosis of claim 4, wherein the position and/or orientation of the pad retainer is infinitely adjustable relative to the lower leg frame.

6. The knee orthosis of claim 2, wherein the calf pad is detachably arranged on the pad retainer.

7. The knee orthosis of claim 1, wherein the at least one elastic bearing element has a compression element, preferably a compression spring and/or a compression element made of an elastic material.

8. The knee orthosis of claim 1, wherein the at least one elastic bearing element has at least one tension element, preferably a tension spring and/or at least one elastic strap.

9. The knee orthosis of claim 2, wherein the calf pad is arranged on the pad retainer such that it can be swiveled about at least a second swivel axis, which is preferably vertical to the first swivel axis.

10. The knee orthosis of claim 1, wherein the knee orthosis features a measurement scale on which a strength of the tension of the at least one elastic bearing element and/or a force generated by this tension can be read.

11. A knee orthosis comprising:
an upper leg frame for arranging on an upper leg;
a lower leg frame for arranging on a lower leg; wherein the lower leg frame is arranged on the upper leg frame such that it can be swiveled about a first swivel axis by means of at least one joint; and
a calf pad arranged on a pad retainer on the lower leg frame for mounting on a calf of a wearer of the knee orthosis, wherein the knee orthosis comprises at least one elastic bearing element that is attached in such a way that the at least one bearing element is tensioned when the knee orthosis is mounted and, in this state, presses the calf pad either directly or indirectly against the calf of the wearer of the knee orthosis, and wherein the pad retainer can be fixed on the lower leg frame in multiple positions and/or orientations relative to the lower leg frame.

12. The knee orthosis of claim 11, wherein the calf pad is arranged on the pad retainer via the at least one elastic bearing element.

13. The knee orthosis of claim 11, wherein the at least one elastic bearing element in the tensioned state exerts a frontal-acting force on the calf pad.

14. The knee orthosis of claim 11, wherein a position and/or orientation of the pad retainer is infinitely adjustable relative to the lower leg frame.

15. The knee orthosis of claim 11, wherein the calf pad is detachably arranged on the pad retainer.

16. The knee orthosis of claim 11, wherein the at least one elastic bearing element has a compression spring.

17. The knee orthosis of claim 11, wherein the at least one elastic bearing element has at least one tension spring and/or at least one elastic strap.

18. The knee orthosis of claim 11, wherein the calf pad is arranged on the pad retainer such that it can be swiveled about at least a second swivel axis, which is preferably vertical to the first swivel axis.

19. The knee orthosis of claim 1, wherein the knee orthosis features a measurement scale on which a strength of the tension of the at least one elastic bearing element and/or a force generated by this tension can be read.

20. A knee orthosis comprising:
an upper leg frame for arranging on an upper leg;
a lower leg frame for arranging on a lower leg; wherein the lower leg frame is arranged on the upper leg frame such that it can be swiveled about a first swivel axis by means of at least one joint; and
a calf pad detachably arranged on a pad retainer on the lower leg frame for mounting on a calf of a wearer of the knee orthosis, wherein a position and/or orientation of the pad retainer is infinitely adjustable relative to the lower leg frame, and wherein the knee orthosis comprises at least one elastic bearing element that is attached in such a way that the at least one bearing element is tensioned when the knee orthosis is mounted and, in this state, presses the calf pad either directly or indirectly against the calf of the wearer of the knee orthosis, and wherein the pad retainer can be fixed on the lower leg frame in multiple positions and/or orientations relative to the lower leg frame.
